(19) United States
(12) Patent Application Publication
(10) Pub. No.: US 2010/0266665 A1
(43) Pub. Date: Oct. 21, 2010

(54) Fenugreek Flour for Incorporating Into Food Products

(76) Inventors: Jack N. Losso, Baton Rouge, LA (US); John W. Finley, Baton Rouge, LA (US); Darryl L. Holliday, Baton Rouge, LA (US); Luke A. Mumphrey, Baton Rouge, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 12/762,896

(22) Filed: Apr. 19, 2010

Related U.S. Application Data
(60) Provisional application No. 61/170,706, filed on Apr. 20, 2009.

(51) Int. Cl. A61K 9/14; A61K 36/48; A61P 5/50; A61P 3/10; A21D 2/36; A21D 13/04; A23L 1/29

(52) U.S. Cl. 424/439; 424/757; 426/460; 426/385; 426/629; 426/550; 426/589; 426/590; 426/599

(57) ABSTRACT

A process for obtaining fenugreek flour from fenugreek seeds has been developed, and the flour incorporated into baked or other food products. The processed fenugreek flour from boiled seeds was easier to add into food products than whole seeds and did not compromise the food's taste or texture. This fenugreek flour can be included in everyday food items and will allow increased use in fenugreek to aid in the prevention of diabetes and obesity. Using this flour, a fenugreek bread formula was developed, and the bread was produced in a commercial bakery by incorporating fenugreek flour into a standard wheat bread formula. Eight diet controlled diabetic subjects were served wheat bread with about 5% fenugreek, and the consumption of the fenugreek bread was shown to reduce serum insulin indicating the bioactivity of the fenugreek remained in the bread.

The benefit of the Apr. 20, 2009 filing date of U.S. provisional application Ser. No. 61/170,706 is claimed under 35 U.S.C. §119(e).

This invention pertains to a method for producing fenugreek flour from fenugreek seeds that has a less bitter flavor but without the use of an organic solvent. This fenugreek flour can be incorporated into food products, such as cookies, cereals, crackers, doughnuts, bagels, biscuits, pizza dough, pasta, bread, other baked goods, juices, salads, gravies, sauces, and candies.

Fenugreek (Trigonella foenum graecum) has been incorporated into many foods, including yeast dough products, condiments, and even as flavoring for imitation maple syrup. See, for example, U.S. Pat. Nos. 4,501,757; 5,264,240 and (1).

Fenugreek has also been used in folk-medicine to treat diabetes mellitus and has been shown to lower blood sugar in both type 1 and type 2 diabetes as well as to improve dyslipidemia. Madar et al. (2) conducted a 3 hour meal tolerance test with or without 15 grams of powdered fenugreek seeds on subjects with type 2 diabetes. They observed a significant reduction in blood glucose, but insulin levels, although lower, were not statistically significantly lower, and there was no change in serum lipids (2). Sharma et al. (3) gave subjects 50 grams of defatted fenugreek powder twice a day for 10 days. Fenugreek reduced fasting glucose significantly and reduced urinary glucose excretion by 54%. Total cholesterol, low density lipoprotein cholesterol (LDL), and triglycerides were also reduced significantly, but high density lipoprotein cholesterol (HDL) was unchanged. Sowmya and Rajyalakshmi (4) fed either 12.5 grams or 18 grams per day of fenugreek powder to 20 hypercholesterolemic adults for 1 month and documented a significant reduction in total and LDL cholesterol, but without change in HDL cholesterol or triglycerides, confirming a hypolipidemic effect of fenugreek in humans.

Gupta et al. (5) conducted a randomized study with 25 type 2 diabetic subjects who exhibited fasting blood glucose levels <200 mg/dL. These diabetic subjects were fed for two months each day either 1 gram of water, 1 g of alcohol extract of fenugreek seeds, or 1 g of a placebo. There was a significant reduction of the area under the curve for glucose and insulin during a 2 hour glucose tolerance test, a significant increase in HDL cholesterol, and a reduction in triglycerides. Fenugreek has also been used in combination with sulfonylureas in the treatment of symptomatic type 2 diabetes. Sixty-nine type 2 diabetic subjects, not well controlled on sulfonylurea drugs alone, were randomized in two groups and consumed three times a day for several weeks either 2 grams of total fenugreek saponins or 2 grams of placebo. There was a significant improvement in diabetic symptoms, fasting glucose, 2 hour post-prandial glucose, and gycosylated hemoglobin (6).

Studies in rodents fed fenugreek have shown a decrease in blood sugar without a change in insulin (8). The explanation for the improvement in blood glucose with both type 1 and type 2 diabetes has been that fenugreek stimulates the insulin signaling pathway. Fenugreek has been shown in rodents to stimulate tyrosine phosphorylation of the insulin receptor, insulin receptor substrate 1, and the p85 subunit of PI3-kinase without having any effect on protein kinase B (Akt). A fenugreek stimulation of the insulin signaling pathway has been demonstrated in rodent adipocytes and liver cells (9).

In addition to stimulating the insulin signaling pathway, fenugreek also inhibits glucose absorption in rodents by inhibiting intestinal disaccharidases (9). An amino acid, 4-hydroxyisoleucine, has been extracted from fenugreek seeds, and shown to decrease glucose and insulin in diabetic rodents, improve glucose tolerance, and stimulate insulin secretion from pancreatic rodent islets (10, 11, 12). Fenugreek seed powder has also been shown to restore altered pyruvate kinase and phosphoenolpyruvate carboxykinase levels, two key enzymes in glycolysis and gluconeogenesis, respectively, in addition to correcting alteration in the distribution of glucose transporter 4 (GLUT-4) in skeletal muscle (13). In fact, low doses of fenugreek and vanadate have been shown to be synergistic in regulating sodium-potassium adenosine triphosphatase activity and GLUT-4 translocation in alloxan diabetic rodents without any harmful side effects (14).

Fenugreek has other beneficial effects in addition to its effect on diabetes. Fenugreek was demonstrated to reduce renal calcification in rodents with ethylene glycol-induced calcium oxalate kidney stones (15). Fenugreek seeds increase swimming time to exhaustion in rodents by increasing utilization of fatty acids (16). In addition, fenugreek seed extract decreases body weight gain on a high fat diet and decreases triglycerides in rodents (17). Fenugreek polyphenols decrease alcoholic fatty liver in rodents (18). Fenugreek seeds reduce hepatic steatosis by a reduction in TNF-alpha (19).

Fenugreek has also been demonstrated to be safe at high doses. Acute and subchronic toxicity assessments of solvent-extracted, debitterized fenugreek powder in mice and rats to doses of 2 and 5 grams per kilogram body weight, the highest doses practical, did not show any significant acute or cumulative toxicity (20).

Despite evidence demonstrating the efficacy of fenugreek in the treatment of diabetes, its use for that purpose has been inhibited by its bitter taste (7). Fenugreek, when incorporated in foods, usually imparts bitterness to the final product, lowers the palatability of the food, and thus limits its use in food. Fenugreek seeds are known to contain a combination of galactomannans, saponins, Bowman-Birk inhibitor, flavonoids, lignins, and 4-hydroxyisoleucine. Various methods have been tried to reduce the bitter flavor, including roasting the seeds and extracting the bitter compounds with alcoholic or petroleum-based solvents. See, for example, U.S. Pat. Nos. 5,264,240 and 5,658,571. U.S. Pat. No. 5,658,571 describes a process for preparing a debitterized powder of the seeds of fenugreek in which the fenugreek does not come in direct contact with steam and alcohols are used to extract the bitter compounds. U.S. Pat. No. 5,997,877 describes a process for the fractionation of fenugreek seeds, resulting in fractions that include a soluble dietary fiber fraction, de-flavored fenugreek seeds, high-protein fenugreek meal, saponins, and oleoresins. U.S. Pat. No. 7,645,466 describes a process to extract amino acid compositions from fenugreek seed. U.S. Published Patent Application No. 2001/0024665 describes a process to extract oils, oleoresin and dietary fiber from fenugreek seed.

We have developed a process for obtaining fenugreek flour from seeds, and incorporating the flour into baked or other food products. This process uses only water and boiling to decrease the bitter flavor from the fenugreek seeds. No alcohol or petroleum-based solvents were used. The process involved boiling the seeds with water, cooling, and then removing the water by drying. The dried seeds are then ground into a fenugreek flour product. This new fenugreek flour was easier to add into food products than whole seeds and did not compromise the food's taste or texture. This fenugreek flour can be included in everyday food items with little to no compromise in taste and will allow increased use of fenugreek to aid in the prevention of diabetes and obesity. Using this flour, we developed a fenugreek bread formula that was produced in a commercial bakery by incorporating fenugreek flour into a standard wheat bread formula. The fenugreek bread was reported by a sensory panel to be indistinguishable from the whole wheat bread control in terms of texture and taste. In a double-blind study, eight diet-controlled, diabetic subjects were served on separate occasions either two slices of bread (56 grams) with 5% fenugreek or of bread without fenugreek, and assayed for serum glucose and insulin levels. The fenugreek bread was shown to lower serum glucose and insulin levels in the diabetic patients, but only the reduction in insulin was significantly different. The fenugreek flour from the boiled seeds still retained some bioactivity.

### BRIEF DESCRIPTION OF DRAWINGS

FIG. 1 illustrates the blood glucose levels over a 4 hr period in diet-controlled diabetic subjects fed fenugreek bread (Fenugreek) and whole wheat bread (Control).

FIG. 2 illustrates the serum insulin levels over a 4 hr period in diet-controlled diabetic subjects fed fenugreek bread (Fenugreek) and whole wheat bread (Control).

A method is described to produce fenugreek flour and use this flour for the preparation of food products, including without limitation, cookies, cereals, crackers, doughnuts, bagels, biscuits, pizza dough, pasta, breads, other baked goods, tortillas, juices, other beverages, salads, gravies, sauces, and candies. Fenugreek flour was added to bread to provide about 1.25 g to 2.5 g fenugreek flour per slice. This amount of powder provided about 5 g fenugreek per day based on one serving, an amount equal to about one-fifth of the suggested total daily intake of fenugreek (24 g/day). Acceptable baked products can be prepared with added fenugreek which will help reduce insulin resistance and aid in treatment of type 2 diabetes.

### EXAMPLE 1

### Preparation of Fenugreek Flour

The fenugreek seeds were mixed with water and boiled for about 4 minutes to inactivate the lectins. The mixture was cooled, and dried by lyophilization. Other drying methods could be used, for example, evaporation. The dried mixture was then ground to a powder consistency. The resulting flour was less viscous and had less odor than flour produced from untreated, ground fenugreek seeds. The treated fenugreek flour was added to a loaf of bread as partial replacement for wheat or other flour, such that a subject fed two slices of the bread would consume about 5 g fenugreek flour per day. The successful use of only water and boiling to remove much of the bitter flavor was a surprising result. Most extraction processes for removal of the bitter flavor of fenugreek have used an alcohol, petroleum-based, or other organic solvent.

A more detailed description of the preparation of the fenugreek seeds follows. The whole, raw fenugreek seeds were mixted with water (a ratio of about 1:3 w/w seed to water) and then boiled for about 3 to 5 min. The mixture was then dried to remove most of the water. In one embodiment, the mixture was cooled, and then dried by lyophilization. Other drying methods could be used, e.g., blast steaming, zeodration, or tumble drying. In addition, the seeds could be cracked prior to boiling to increase the surface area and decrease the drying time (if the cracking did not result in excessive water gain). At the end of the drying process, the dried seeds were ground using a mill, but other size-reduction methods could be used. The grinding continued until about 90% or more of the mixture by weight had particles with a diameter less than about 250 μm and passed through a 60 mesh screen. The “diameter” of a particle refers to the longest dimension across or through the particle, and does not imply that the particle has any particular shape. The resulting powder with a consistency of flour weighed as much as the original fenugreek seeds, but had less viscosity and a less intense flavor than flour produced from a similar grinding of raw (not boiled) fenugreek seeds.

Different levels of fenugreek flour (from none to about 12 g, 50% of the suggested total daily intake) were added to bread dough. In one experiment, a white flour bread without any fenugreek, a white bread prepared with treated fenugreek flour (about 0.975 g per slice), and a white bread prepared with untreated fenugreek powder were made. The bread with the treated fenugreek flour did not behave as that with the raw fenugreek flour. The raw fenugreek flour inhibited leavening, and the bread product was grainy. Raw fenugreek flour was very viscous and competed with the regular flour for water, resulting in a flat bread. Also the yeast appeared to lose its viability in the presence of raw fenugreek flour. In contrast, by pre-cooking the seeds and drying prior to grinding, the treated fenugreek flour was less viscous with a less intense flavor. These three bread products were tested using a sensory panel of eight adults. The sensory panel noticed a slight maple-like flavor and sweetness when the fenugreek flour was added at low levels (about 0.975 g per slice) to the white bread dough. Similarly, the treated fenugreek flour was added to bread that used 50% whole wheat flour and bread that used 100% whole wheat flour (dark wheat). In each case, the taste of the treated fenugreek flour was lessened by the addition of wheat flour. The bitter flavor was noticed less in the stronger flavored bread. The bitter flavor would also be noticed less in already bitter breads, e.g., rye, or pumpernickel, and thus higher amounts of treated fenugreek flour (e.g., up to 7.5 g per serving size) could be incorporated into these darker breads. The treated fenugreek flour was also incorporated into cookie dough (chocolate chip and oatmeal raisin) using standard recipes such that 0.1 g fenugreek flour was added to each 18 g cookie dough. There was no taste difference noted in the cookies from the addition of fenugreek as compared to cookies without the fenugreek powder. The fenugreek powder was also incorporated into both corn and flour tortilla mixtures, 0.8 g fenugreek powder per 48 g tortilla dough. Again, there was no difference in flavor due to the fenugreek powder in the resulting tortillas.

### EXAMPLE 2

### Effect of Fenugreek Flour on Bread Composition and Acceptability

A bread formulation containing the treated fenugreek flour was developed in the laboratory as described above and then adapted for production in a commercial bakery. The formulation incorporated fenugreek flour prepared as above into a standard wheat bread formula by replacing about 9% wheat flour with fenugreek flour. The fenugreek flour was added at amounts of 25 to 50 g per loaf to the other bread ingredients. One loaf weighed approximately 527 g and provided 20 slices. Each slice contained about 1.25 to 2.5 g fenugreek, and one serving portion of two slices contained 2.5 to 5 g fenugreek. The bread ingredients were mixed and the dough was kneaded in a Commercial 5 Series Model #: KM25G0X KitchenAid Mixer (KitchenAid Portable Appliance, St. Joseph, Mich.) using a McDuffy mixing bowl. The bread was proofed at 43.3° C. at 85% humidity for 65 minutes in a Proofing Cabinet (Hobart, Troy, Ohio), and then baked at 221.1° C. for 21 min. Similarly whole wheat bread controls were prepared following the same wheat bread formula (but without the fenugreek substitution), and using the same equipment and preparation method. All bread samples were stored at −20° C. until use. Bread samples were analyzed for macronutrients, namely moisture, crude protein, fat, carbohydrate, and ash.

Sensory evaluation of the bread included rating the bread for palatability, flavor, texture, and color change. Ten individuals evaluated the fenugreek bread and compared it to the control whole wheat bread. The panelists were a mixture of untrained persons, persons with previous sensory evaluation experience, and persons both with and without diabetes. The bread was presented to panelists either at their offices or homes, and each panelist was asked to evaluate the appearance, color, flavor, texture, and overall acceptability.

The proximate compositions of whole wheat and fenugreek bread samples are shown in Table 1. Results in Table 1 indicate that no significant differences were found between the fenugreek bread and the whole wheat control bread in moisture, fat, carbohydrate or protein content.

TABLE 1
Proximate Composition of Wheat and Fenugreek Bread

| Composition (%) | Wheat Bread | Fenugreek Bread |
|---|---|---|
| Moisture | 39.22 ± 1.10 | 40.12 ± 0.96 |
| Carbohydrate^{a} | 82.20 ± 1.26 | 84.12 ± 0.66 |
| Fat^{a} | 2.01 ± 0.03 | 2.02 ± 0.04 |
| Protein^{a} | 20.45 ± 1.01 | 21.57 ± 1.03 |

^{a}Expressed on a dry weight basis. Values are means ± SD of three determinations.

Ten (10) panelists were used in the evaluation of the fenugreek bread. They varied in age, gender, and economic class as well as diabetic status. All consumers stated that they ate bread at least four (4) times per week. They evaluated the bread on its acceptability and palatability. There was no significant differences in color, firmness, texture, and flavor intensity between the fenugreek and whole wheat bread (p<0.05). The rating results are shown in Table 2.

TABLE 2
Results of Bread Sensory Evaluation
(Hedonic rating averages)

| | Wheat Bread | Fenugreek Bread |
|---|---|---|
| Color | 5.36 | 6.00 |
| Firmness | 4.91 | 5.00 |
| Texture | 4.73 | 4.55 |
| Flavor/Intensity | 4.73 | 5.18 |

### EXAMPLE 3

### Effect of Fenugreek Flour on Carbohydrate Metabolism

Eight diet-controlled, type 2 diabetic subjects between the ages of 18 and 70 years who were not pregnant or nursing a child were enrolled for the clinical study. Subjects taking a medication for diabetes or a systemic glucocorticoid that could affect blood sugar were excluded, as were subjects who were taking any chronic medication who had not taken a stable dose for at least one month prior to entering the study.

Subjects had an initial screening visit where they completed a health questionnaire, reviewed it with the study coordinator, and signed an informed consent approved by the local Institutional Review Board. The subjects then returned on two mornings a week, after having nothing but water from 9:00 pm the prior evening. On each of these visits, an intravenous line was placed, and blood drawn for testing of glucose and insulin at −30, −15, 0, 15, 60, 120, 180 and 240 minutes. Time 0 corresponded to the eating of the two slices of bread. On one visit a subject ate two slices of bread each containing 2.5 grams of fenugreek flour, and on the other visit the same subject was given the control bread. All subjects were given both fenugreek and control bread by the end of the study. The two bread conditions were double-blinded and the order of giving the bread conditions was randomized. Subjects were questioned about any adverse events during their testing days. The results were analyzed as area under the curve for glucose and insulin and were compared by paired t-tests.

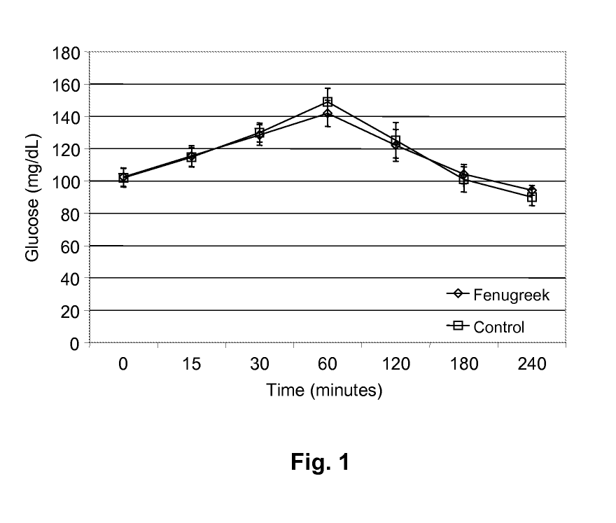

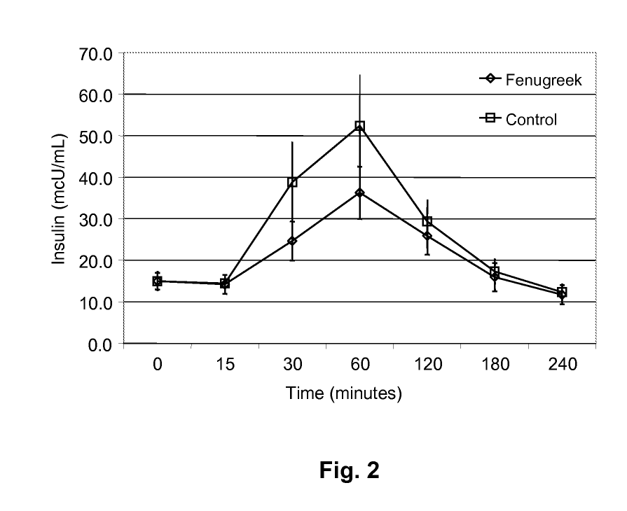

All 8 subjects completed the study. Subject characteristics are described in Table 3, in which the data are shown as mean (SD) values where possible. The −30, −15 and 0 time points were averaged to give a stable baseline. FIG. 1 shows the average blood glucose for the subjects under the two conditions. In FIG. 1, the area under the curve for serum glucose was 499 mg/dl-min less for the fenugreek flour bread than for the control wheat bread (no fenugreek flour), but the standard deviation was high, 2439 mg/dl-min, and the differences not significant (p=NS, FIG. 1). FIG. 2 shows the amount of insulin in the serum. The area under the curve for insulin was 1650 mcU/ml-min less for the fenugreek flour bread than for the control wheat bread (without fenugreek flour) with a standard deviation of 1650 mcU/ml-min, and the differences were significant (p<0.05, FIG. 2). The area under the curves for both glucose and insulin was lower in the patients given the fenugreek bread, but the difference between intake of the two breads was only statistically significant (p<0.05) in the serum insulin results. There were no adverse events from eating the fenugreek flour bread reported by the study subjects.

TABLE 3
Demographic Data for Study Subjects

| Characteristic | Value | |
|---|---|---|
| Total number of subjects | 8 | |
| Race | | |
| Caucasian | 4 | |
| Black | 4 | |
| Gender | | |
| Male | 2 | |
| Female | 6 | |
| Age (years) | 60.0 | (6) |
| Weight (kg) | 93.0 | (13.41) |
| Body Mass Index | 32.9 | (4.85) |
| Systolic blood pressure | 115 | (9.1) |
| Diastolic blood pressure | 73 | (7.6) |
| Heart rate | 72 | (8.0) |

The flour from the boiled fenugreek seeds was shown to be palatably incorporated into baked goods while retaining a beneficial effect on serum insulin in diet-controlled, type 2 diabetic subjects. Fenugreek flour incorporation into baked goods may offer a safe and effective food to aid in the treatment of type 2 diabetes and to reduce adverse effects associated with insulin resistance.

The term “effective amount” as used herein refers to an amount of the treated fenugreek flour orally given by incorporating into a food product in which the amount is sufficient to lower the blood glucose or the blood insulin to a statistically significant degree (p<0.05). The dosage ranges for the addition of the fenugreek powder to the food product include those that produce the desired effect. Generally, the dosage will vary with the age, weight, condition, sex of the patient or pathology, the resulting palatability of the food product, and the degree of desired effect. A potential dose as shown above is from 2.5 to 5.0 g treated fenugreek flour per day. A person of ordinary skill in the art, given the teachings of the present specification, may readily determine suitable amounts to be added to food products. In any event, the effectiveness of treatment can be determined by monitoring the blood glucose or blood insulin by methods well known to those in the field.

### REFERENCES

1. Bartley G B, Hilty M D, Andreson B D, Clairmont A C, Maschke S P. Maple-syrup urine odor due to fenugreek ingestion. N Engl J Med. 305(8):467, 1981.
2. Madar Z, Abel R, Samish S, Arad J. Glucose-lowering effect of fenugreek in non-insulin dependent diabetics. Eur J Clin Nutr. 42(1):51-54, 1988.
3. Sharma R D, Raghuram T C, Rao N S. Effect of fenugreek seeds on blood glucose and serum lipids in type I diabetes. Eur J Clin Nutr. 44(4):301-306, 1990.
4. Sowmya P, Rajyalakshmi P. Hypocholesterolemic effect of germinated fenugreek seeds in human subjects. Plant Foods Hum Nutr. 53(4):359-365, 1999.
5. Gupta A, Gupta R, Lal B. Effect of Trigonella foenum-graecum (fenugreek) seeds on glycaemic control and insulin resistance in type 2 diabetes mellitus: a double blind placebo controlled study. J Assoc Physicians India. 49:1955-1056, 2001.
6. Lu F R, Shen L, Qin Y, Gao L, Li H, Dai Y. Clincal observation on Trigonella foenum-gracum L. total saponins in combination with sulfonylureas in the treatment of type 2 diabetes mellitus. Chin J Integr Med. 14(1):56-60, 2008
7. Pathak P, Srivastava S, Grover S. Development of food products based on millets, legumes and fenugreek seeds and their suitability in the diabetic diet. Int J Food Sci Nutr. 51(5):409-414, 2000.
8. Hannan J M, Ali L, Rokeya B, Khaleque J, Akhter M, Flatt P R, Abdel-Wahab Y H. Soluble dietary fibre fraction of Trigonella foenum-graecum (fenugreek) seed improves glucose homeostasis in animal models of type 1 and type 2 diabetes by delaying carbohydrate digestion and absorption, and enhancing insulin action. Br J Nutr. 2007 Mar; 97(3):514-21.
9. Vijayakumar M V, Singh S, Chhipa R R, Bhat M K. The hypoglycaemic activity of fenugreek seed extract is mediated through the stimulation of an insulin signaling pathway. Br J Pharamcol. 146(1):41-48, 2005.
10. Broca C, Gross R, Petit P, Sauvaire Y, Manteghetti M, Tournier M, Masiello P, Gomis R, Ribes G. 4-hydroxyisoleucine: experimental evidence of its insulinotropic and antidiabetic properties. Am J Physiol Endocrinol Metab 277:617-623, 1999.
11. Broca C Manteghetti M, GrossR, Baissac Y, Jacob M, Petit P, Sauvaire Y, Ribes G. 4-Hydroxyisoleucine : effects of synthetic and natural analogues on insulin secretion. Eur J Pharmacol. 390(3):339-345, 2000.
12. Broca C, Breil V, Cruciani-Guglielmacci, C, Manteghetti M, Roualt C, Derouet M, Rizkalla S, Pau B, Petit P, Ribes G, Ktorza A, Gross R, Reach G, Taouis M. Insulinotropic agent ID-1101 (4-hydroxyisoleucine) activates insulin signaling in rat. Am J Physiol Endocrinol Metab. 287:E463-E471, 2004.
13. Mohammad S, Taha A, AkhtarK, Bamezai RN, Baquer N Z. In vivo effect of Trigonella foenum graceum on the expression of pyruvate kinase, phosphoenolpyruvate carboxykinase, and distribution of glucose transporter (GLUT4) in alloxan-diabetic rats. Can J Physiol Pharmacol. 84(6):647-654, 2006.
14. Siddiqui M R, Moorthy K, Taha A, Hussain M E, Baquer N Z. Low doses of vanadate and Trigonella synergistically regulate Na⁺/K⁺ ATPase activity and GLUT4 translocation in alloxan-diabetic rats. Mol Cell Biochem. 285(1-2):17=27, 2006.
15. Laroubi A, Touhami M, Farouk L, Zrara I, Aboufatima R, Benharref A, Chait A. Prophylaxis effect of Trigonella foenum graecum L. seeds on renal stone formation in rats. Phytother Res. 21(10):921-925, 2007.
16. Ikeuchi M, Yamaguchi K, Koyama T, Sono Y, Yazawa K. Effects of fenugreek seeds (Trigonella foenum greacum) extract on endurance capacity in mice. J Nutr Sci Vitaminol. 52:287-292, 2006.
17. Handa T, Yamaguchi K, Sono Y, Yazawa K. Effects of fenugreek seed extract in obese mice fed a high-fat diet. Biosci Biotechnol Biochem. 69(6):1186-1188, 2005.
18. Kaviarasan S, Sundarapandiyan R, Anuradha C V. Protective action of fenugreek (Trigonella foenum graecum) seed polyphenols against alcohol-induced protein and lipid damage in rat liver. Cell Biol Toxicol. [Epub ahead of print], 2008.
19. Raju J, Bird R P. Alleviation of hepatic steatosis accompanied by modulation of plasma and liver TNF-alpha levels by Trigonella foenum graecum (fenugreek) seeds in Zucker obese (fa/fa) rats. Int J Obes (Lond). 30(8):1298-12307, 2006.
20. Muralidhara, Narasimhamurthy K, Viswanatha S, Ramesh B S. Acute and subchronic toxicity assessment of debitterized fenugreek powder in the mouse and rat. Food Chem Toxicol. 37(8):831-838, 1999.

The complete disclosures of all references cited in this application are hereby incorporated by reference. The complete disclosure of the following is incorporated: J.N. Losso et al., “Fenugreek bread: A treatment for diabetes mellitus”, Journal of Medicinal Foods, vol. 12(5), pp. 1046-1049 (2009). In the event of an otherwise irreconcilable conflict, however, the present specification shall control.

We claim:

1. A process for making modified fenugreek flour, said process comprising the steps of:
(a) mixing raw fenugreek seeds with water;
(b) heating the mixture of fenugreek seeds and water to a temperature about 100° C. or higher, for at time about 3 minutes or higher;
(c) drying the mixture to remove most of the water and moisture; and
(d) grinding the dried mixture until about 90% or more of the ground mixture by weight are particles having a diameter less than about 250 μm;
wherein:
(e) said process employs no added organic solvent; and
(f) the modified fenugreek flour made by said process is substantially less bitter than otherwise identical raw fenugreek seeds would be, if the raw fenugreek seeds were directly ground to the same size as the modified fenugreek flour.

2. The process of claim 1, wherein said heating step comprises boiling the mixture of fenugreek seeds and water for about 5 minutes.

3. The process of claim 1, wherein the ratio of seeds to water prior to said heating step is about 1 part of seeds to 3 parts water on a weight basis.

4. The process of claim 1, wherein said drying step comprises evaporation at atmospheric pressure.

5. The process of claim 1, wherein said drying step comprises freeze-drying.

6. The modified fenugreek flour produced by the process of claim 1, wherein said modified fenugreek flour is substantially less bitter than otherwise identical raw fenugreek seeds would be, if the raw fenugreek seeds were directly ground to the same size as the modified fenugreek flour

7. A food product comprising the modified fenugreek flour of claim 6; wherein said food product is substantially less bitter than an otherwise identical food product would be, if the otherwise identical food product were prepared with the same amount of ground, raw fenugreek seeds.

8. The food product of claim 7, wherein said food product is selected from the group consisting of pasta, breads, bagels, pizza dough, doughnuts, pastries, candies, breakfast cereals, cookies, cakes, crackers, biscuits, other baked goods, tortillas, juices, other beverages, salads, gravies, sauces, and candies.

9. The food product of claim 7, wherein said food product is bread.

10. A method to reduce insulin resistance in a diabetic patient, said method comprising administering to the patient an effective amount of the food product of claim 7.

11. The method of claim 10, wherein the food product is bread.

12. The method of claim 10, wherein the amount of the fenugreek flour administered in the food product is an amount equal to about 2.5 to about 5.0 grams daily.
